(19) United States
(12) Patent Application Publication
(10) Pub. No.: US 2016/0287849 A1
(43) Pub. Date: Oct. 6, 2016

(54) VIBRATING SELF-DILATION BOUGIE

(71) Applicant: Boston Scientific Scimed, Inc., Maple Grove, MN (US)

(72) Inventor: Megan Hodson, Maple Grove, MN (US)

(21) Appl. No.: 15/087,905

(22) Filed: Mar. 31, 2016

Related U.S. Application Data
(60) Provisional application No. 62/141,040, filed on Mar. 31, 2015.

(51) Int. Cl. A61M 29/00

(52) U.S. Cl. CPC A61M 29/00; A61M 2210/105; A61B 2017/22021

(57) ABSTRACT

A dilation device, comprises an elongated member extending longitudinally from a proximal end to a distal end, the elongated member sized and shaped to be received within a target area of an esophageal passage to dilate the target area, a projection extending along the elongated member from an outer surface thereof so that, when the elongated member is received within the target area, the projection extends into a wall of the esophageal passage, and a vibration mechanism which, when activated, vibrates one of the elongated member and the projection to break away a cell blockage within the target area.

### CROSS-REFERENCE TO RELATED APPLICATIONS

This application claims priority under 35 U.S.C. §119(e) to co-pending U.S. Provisional Application Ser. No. 62/141,040, filed on Mar. 31, 2015, herein incorporated by reference in its entirety.

### BACKGROUND

Esophageal cancer may result in cell or tissue growth which narrows the esophageal passage making it difficult to perform daily activities such as swallowing. In some cases, treatment options may be limited and/or a patient may have difficulty maintaining nutrition during treatment due to dysphagia. In these cases, a dilation bougie may be inserted into the patient's esophagus to temporarily dilate the esophageal passageway, relieving discomfort due to dysphagia and allowing the patient to maintain nutrition via the oral intake of food.

### SUMMARY

In one implementation, a medical device that may include an elongated member extending longitudinally from a proximal end to a distal end, the elongated member sized and shaped to be received within a target area of an esophageal passage to dilate the target area. The dilation device may also include a projection extending along the elongated member from an outer surface thereof so that, when the elongated member is received within the target area, the projection extends into a wall of the esophageal passage. The dilation device may further include a vibration mechanism which, when activated, vibrates one of the elongated member and the projection to break away a cell blockage within the target area.

In an embodiment, the projection may extend helically about the elongated member along a portion of a length thereof.

In an embodiment, the projection may include a plurality of rings extending about the elongated member along a portion of a length thereof.

In an embodiment, the projection may include a plurality of hemispherical protrusions.

In an embodiment, the projection may be integrally formed with the elongated member.

In an embodiment, the elongated member may be formed of a flexible rubber material.

In an embodiment, the distal end of the elongated member may be tapered.

In an embodiment, the proximal end of the elongated member may include an activation element for activating the vibration mechanism.

In an embodiment, the vibration mechanism includes a motor housed within the elongated member.

In an embodiment, the vibration mechanism may include a wire extending through the projection for receiving a high frequency current.

In one implementation, a device for dilating a passage that may include a flexible elongated member extending longitudinally from a proximal end to a tapered distal end, the elongated member sized and shaped to be inserted through a passage. The device may further include a projection extending helically about the elongated member along at least a distal portion thereof and a vibration mechanism housed within one of the elongated member and the projection so that, when the vibration mechanism is activated, the elongated member vibrates to dilate the passage and break away a cell blockage therein.

In an embodiment, one of the elongated member and the projection may be formed of a rubber material.

In an embodiment, the projection may be integrally formed with elongated member.

In an embodiment, the vibration mechanism may include a motor configured to be activated via an activation element at the proximal end of the elongated member.

In an embodiment, the vibration mechanism may include a wire extending through the projection, the wire configured to receive a high frequency current therethrough.

In one implementation, a method for dilation that may include placing a distal end of an elongated member trans-orally to a target area within an esophagus. The method may also include activating a vibrating mechanism housed within the elongated member so that a projection extending from an outer surface of the elongated member vibrates against a wall of the target area to break away a blockage formed thereon.

### BRIEF DESCRIPTION OF DRAWINGS

FIG. 1 shows a partially transparent longitudinal side view of a distal portion of a device according to an exemplary embodiment of the present invention;

FIG. 2 shows a partially transparent longitudinal side view of a distal portion of a device according to an alternate embodiment of the present invention; and

FIG. 3 shows a partially transparent longitudinal side view of a distal portion of a device according to another alternate embodiment of the present invention.

### DETAILED DESCRIPTION

The present disclosure may be further understood with reference to the following description and the appended drawings, wherein like elements are referred to with the same reference numerals. The present disclosure is directed to a device for dilating a bodily passage and, in particular, to a device for dilating an esophagus. Exemplary embodiments of the present disclosure describe a dilation device sized and shaped to be inserted into an esophagus of a patient and including a vibration mechanism which vibrates the dilation device to aid in breaking up a cell blockage in the esophagus to make it easier for the patient to perform daily activities such as swallowing. The vibration mechanism prolongs the effectiveness of the dilation, so that the dilation procedure does not have to be performed as frequently. It should be noted that the terms “proximal” and “distal,” as used herein, are intended to refer to a direction toward (proximal) and away from (distal) a user (e.g., physician) of the device.

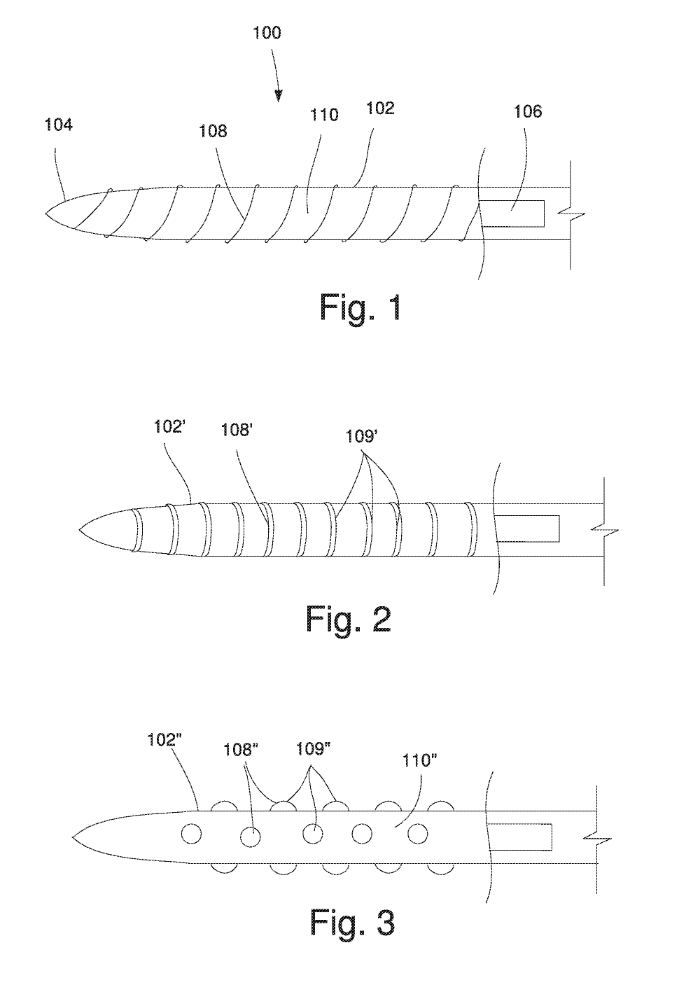

As shown in FIG. 1, a device 100 for dilating an esophageal passage of a patient comprises an elongated member 102 and a vibration mechanism 106 housed within the elongated member 102. The elongated member 102 according to this embodiment is sized and shaped to be inserted into an esophagus of the patient and is formed of a material having sufficient flexibility so that a distal end 104 of the elongated member 102 may be inserted trans-orally to be positioned in a target area within a desired portion of the esophagus. The device 100 also comprises a projection 108 extending about the elongated member 102 from an outer surface 110 of thereof so that, when the elongated member 102 is inserted to the target area, the projection 108 presses against a wall of the esophagus. Upon activation of the vibration mechanism 106, the projection 108 both dilates the esophagus and breaks up cell blockage in the target area. The device 100 provides a minimally invasive, low-cost, reusable therapy to allow patients to maintain nutrients via oral intake during treatment or palliation periods.

The elongated member 102 extends longitudinally from a proximal end (not shown) to the distal end 104. The distal end 104 may optionally be tapered to facilitate oral insertion thereof to the target area within the esophagus. The elongated member 102 may be formed of any of a variety of materials having sufficient flexibility to permit the elongated member 102 to be navigated through the esophagus via the oral cavity. In one example, the elongated member 102 is formed of silicone or another similar biocompatible rubber material such as, for example, polyamide, polyether block amide, polyethylene, polyethylene terephthalate, polyvinylchloride, polyurethane, polytetrafluoroethylene, and copolymers. A diameter of the distal portion of the elongated member 102 is selected to be larger than that of the target area of the esophagus so that insertion of the distal portion of the elongated member 102 to the target area results in a dilation thereof.

In one exemplary embodiment, as shown, the projection 108 is attached to the outer surface 110 of the elongated member 102 and extends circumferentially about the elongate member 102 in a substantially helical pattern. The projection 108, however, is not required to extend helically about the elongated member 102 and may take any of a variety of shapes and patterns so long as the projection extends radially outward from the outer surface 110 of the elongated member 102 to contact/press against the wall of the target area of the esophagus and aid in the dilation thereof. For example, as shown in FIG. 2, a projection 108′ may comprise one or more rings 109′ extending about an elongated member 102′. Where a plurality of rings 109′ are included in the projection 108′ they may be separated from one another along a length of the distal portion of the elongated member 102′ to ensure contact with the target tissue. In another example, as shown in FIG. 3, a projection 108″ may comprise a plurality of hemispherically shaped protrusions 109″ extending from an outer surface 110″ of an elongated member 102″. Edges of the projection 108 may be rounded to reduce trauma to the esophageal wall when vibrating. The projection 108 may be formed of the same material as the elongated member 102. In one embodiment, the projection 108 may be integrally formed with the elongated member 102 while in another embodiment the projection 108 may be a separate element coupled to the elongated member 102.

The vibration mechanism 106 may be housed within the elongated member 102 or within the projection 108 itself so that, upon activation thereof, at least the distal end 104 of the elongated member 102, which is positioned at the target area within the esophagus, vibrates. In some cases (e.g., when a patient self dilates), the target area may be difficult to locate such that it may be desired to include the vibration mechanism 106 in the elongated member 102 to vibrate an entire length of the elongated member 102 to prolong a dilation of a full length of the esophagus. The vibration mechanism 106 may include any of a variety of actuating elements which vibrate upon activation thereof. For example, the vibration mechanism 106 may include a motor and an unbalanced weight coupled thereto so that, upon activation of the motor, oscillation of the unbalanced weight vibrates the distal portion of the elongated member 102. In another example, the vibration mechanism 106 may include a small wire may be placed in the projections 108 such that upon activation of the vibration mechanism 106, a high frequency current, which would be converted into mechanical vibrations, would travel down the wire. The wire may be formed of, for example, stainless steel, platinum, nickel-chromium alloys, cobalt-chromium alloys, etc. The vibration mechanism 106 may be activated by a button or switch at the proximal end of the elongated member 102. The vibration mechanism 106 causes the elongated member 102 to vibrate at a frequency selected so that the projection 108 may break up cells forming a blockage in the target area of the esophagus. For example, the frequency should be in the range of 15,000 to 20,000 cycles a second/Hz.

According to one exemplary embodiment, a method of using the device 100 comprises inserting the elongated member 102 through the oral cavity into the esophagus until the distal end 104 is positioned in the target area within the esophagus. In this position, the outer surface 110 of the elongated member 102 and the projection 108 contact/press against the wall of the esophagus adjacent to the cells forming the blockage. When the device is positioned as desired in the target area, the vibration mechanism 106 is activated via, for example, a switch or button at the proximal end of the elongated member 102. The vibration of the projection 108 extending along the elongated member 102 breaks up any cell blockage within target area prolonging the efficacy of the dilation so that the esophagus remains passable for nutrition for a longer time and increasing the time the patient may go between procedures. Once the target area has been sufficiently dilated and/or the cell blockage has been reduced, the vibration mechanism 106 may be deactivated and the device 100 may be removed from the esophagus. The device 100 may be reused, as necessary, to provide dilation during subsequent periods of time.

The method described above may be performed by a variety of operators, including, but not limited to, a physician, a technician, a nurse, a physician assistant, etc. In another embodiment, the patient may be able to perform the above-described procedure on his or herself, without additional assistance, using a numbing agent to numb the esophagus.

While embodiments have been described above, a number of modifications and changes may be made without departing from the scope of the disclosure. Thus, it is intended that the present disclosure cover modifications and variations provided that they come within the scope of the appended claims and their equivalents.

What is claimed is:

1. A medical device, comprising:
an elongated member extending longitudinally from a proximal end to a distal end, the elongated member sized and shaped to be received within a target area of an esophageal passage to dilate the target area;
a projection extending along the elongated member from an outer surface thereof so that, when the elongated member is received within the target area, the projection extends into a wall of the esophageal passage; and
a vibration mechanism which, when activated, vibrates one of the elongated member and the projection to break away a cell blockage within the target area.

2. The device of claim 1, wherein the projection extends helically about the elongated member along a portion of a length thereof.

3. The device of claim 1, wherein the projection includes a plurality of rings extending about the elongated member along a portion of a length thereof.

4. The device of claim 1, wherein the projection includes a plurality of hemispherical protrusions.

5. The device of claim 1, wherein the projection is integrally formed with the elongated member.

6. The device of claim 1, wherein the elongated member is formed of a flexible rubber material.

7. The device of claim 1, wherein the distal end of the elongated member is tapered.

8. The device of claim 1, wherein the proximal end of the elongated member includes an activation element for activating the vibration mechanism.

9. The device of claim 1, wherein the vibration mechanism includes a motor housed within the elongated member.

10. The device of claim 1, wherein the vibration mechanism includes a wire extending through the projection for receiving a high frequency current.

11. A device for dilating a passage, comprising:
a flexible elongated member extending longitudinally from a proximal end to a tapered distal end, the elongated member sized and shaped to be inserted through a passage;
a projection extending helically about the elongated member along at least a distal portion thereof; and
a vibration mechanism housed within one of the elongated member and the projection so that, when the vibration mechanism is activated, the elongated member vibrates to dilate the passage and break away a cell blockage therein.

12. The device of claim 11, wherein one of the elongated member and the projection is formed of a rubber material.

13. The device of claim 11, wherein the projection is integrally formed with elongated member.

14. The device of claim 11, wherein the vibration mechanism includes a motor configured to be activated via an activation element at the proximal end of the elongated member.

15. The device of claim 11, wherein the vibration mechanism includes a wire extending through the projection, the wire configured to receive a high frequency current therethrough.

16. A method for dilation, comprising:
placing a distal end of an elongated member trans-orally to a target area within an esophagus; and
activating a vibrating mechanism housed within the elongated member so that a projection extending from an outer surface of the elongated member vibrates against a wall of the target area to break away a blockage formed thereon.

17. The method of claim 16, wherein the projection extends helically about the elongated member along at least a distal portion thereof.

18. The method of claim 16, wherein the projection is comprised of a plurality of rings extending about an along at least a distal portion of the elongated member.

19. The method of claim 16, further comprising deactivating the vibration mechanism and removing the elongated member from the esophagus.

20. The method of claim 16, wherein the elongated member is formed of a flexible rubber material.
